(19) United States
(12) Patent Application Publication
(10) Pub. No.: US 2024/0393480 A1
(43) Pub. Date: Nov. 28, 2024

(54) RADIATION DETECTION SYSTEM, CONTROL DEVICE, AND INFORMATION PROCESSING APPARATUS

(71) Applicant: CANON KABUSHIKI KAISHA, Tokyo (JP)

(72) Inventors: HIROTO UENO, Kanagawa (JP); TOMOYA SUZUKI, Tokyo (JP); HIROKAZU OHGURI, Chiba (JP); HIDEAKI MORITA, Tokyo (JP); ASATO KOSUGE, Kanagawa (JP); YOSHIAKI SERIZAWA, Kanagawa (JP)

(21) Appl. No.: 18/670,050

(22) Filed: May 21, 2024

(30) Foreign Application Priority Data
May 23, 2023 (JP) 2023-084772
Feb. 7, 2024 (JP) 2024-017342

(51) Int. Cl. G01T 1/02; G01T 1/20

(52) U.S. Cl. CPC G01T 1/023; G01T 1/20182

(57) ABSTRACT

A radiation detection system that performs stop control to stop radiation irradiation of a radiation generation apparatus, based on a result of dose detection performed by a radiation detection apparatus, the radiation detection system involving a communication between the radiation detection apparatus and the radiation generation apparatus via a network in an execution of the stop control, the radiation detection system includes a notification device configured to notify a user of information, and one or more controllers configured to function as determining a communication state of the network, and issuing a predetermined notification based on a result of the determination, wherein the notification device notifies of notification information for the stop control, based on the predetermined notification.

### BACKGROUND OF THE DISCLOSURE

### Field of the Disclosure

The present disclosure relates to a radiation imaging system, a radiation imaging apparatus, a radiation generation apparatus, a communication control apparatus, and an information processing apparatus.

### Description of the Related Art

Some radiation imaging systems are equipped with a flat panel detector (FPD) formed of a semiconductor material as a radiation imaging system that is used in medical image diagnosis and non-destructive inspection using radiation, such as X-ray. In medical image diagnosis, for example, a radiation imaging system is used as a digital radiation imaging system for still image capturing, such as general imaging, and moving image capturing, such as fluoroscopic imaging.

In recent years, studies have been conducted on a multi-function radiation imaging system. Examples of the multi-function include monitoring an emitted radiation dose (cumulative dose) and stopping radiation irradiation in response to the cumulative dose reaching a threshold value (for example, by outputting an irradiation stop signal to stop radiation irradiation to a radiation generation apparatus). This function is referred to as an automatic exposure control (AEC) function. Examples of the AEC function include a method for monitoring an emitted radiation dose by using some of image forming pixels and a method for monitoring an emitted radiation dose by using a separate built-in sensor.

In the automatic exposure control function of a radiation imaging system, accuracy of stopping radiation irradiation is dependent on a communication environment in the radiation imaging system. For example, if the communication environment in the radiation imaging system is not good, and a communication delay occurs in comparison with a communication in a normal communication state, a time period until when a radiation stop signal reaches a radiation generation apparatus increases in the automatic exposure control function, which causes a delay in stopping the radiation irradiation of the radiation generation apparatus.

A radiation imaging system discussed in Japanese Patent Application Laid-Open No. 2005-6829 transfers a communication test image before image capturing, to detect a system state that causes an image generation failure or an image transfer failure, and in response to the detection, image capturing is disabled. While a communication environment that causes an image transfer failure is detectable with the method discussed in Japanese Patent Application Laid-Open No. 2005-6829, an improvement is still needed in the issue of a delay in a radiation irradiation stop instruction during image capturing using the automatic exposure control function.

### SUMMARY OF THE DISCLOSURE

The present disclosure is directed to providing a radiation imaging system capable of detecting a communication environment unsuitable for image capturing using the automatic exposure control function.

According to an aspect of the present disclosure, a radiation detection system that performs stop control to stop radiation irradiation of a radiation generation apparatus, based on a result of dose detection performed by a radiation detection apparatus, the radiation detection system involving a communication between the radiation detection apparatus and the radiation generation apparatus via a network in an execution of the stop control, the radiation detection system includes a notification device configured to notify a user of information, and one or more controllers configured to function as determining a communication state of the network, and issuing a predetermined notification based on a result of the determination, wherein the notification device notifies of notification information for the stop control, based on the predetermined notification.

Further features of the present disclosure will become apparent from the following description of exemplary embodiments with reference to the attached drawings.

### BRIEF DESCRIPTION OF THE DRAWINGS

FIG. 1 illustrates an example configuration of a radiation imaging system.

FIG. 2 illustrates a hardware configuration example of a radiation imaging apparatus of the radiation imaging system.

FIG. 3 illustrates a configuration example of a control unit of the radiation imaging apparatus of the radiation imaging system according to a first exemplary embodiment.

FIG. 4 is a flowchart illustrating a procedure in which the radiation imaging apparatus notifies a user that a communication state is unsuitable for image capturing using the automatic exposure control function, in the radiation imaging system according to the first exemplary embodiment.

FIG. 5 illustrates relations between an imaging enabled/disabled state of the radiation imaging apparatus, a communication measurement period of a communication state estimation unit, and a user notification enabled period of a communication state notification unit, in the radiation imaging system according to the first exemplary embodiment.

FIG. 6 illustrates a configuration example of a radiation generation apparatus in a radiation imaging system according to a second exemplary embodiment.

FIG. 7 is a flowchart illustrating a procedure in which the radiation generation apparatus changes a content of a notification to a user in accordance with a communication state, in the radiation imaging system according to the second exemplary embodiment.

FIG. 8 illustrates a configuration example of a communication control apparatus in a radiation imaging system according to a third exemplary embodiment.

FIGS. 9A and 9B are flowcharts illustrating a procedure in which the communication control apparatus notifies of a communication state unsuitable for image capturing using the automatic exposure control function, in the radiation imaging system according to the third exemplary embodiment.

FIG. 10 illustrates a configuration of a control unit of a radiation imaging apparatus according to a fourth exemplary embodiment.

FIG. 11 illustrates combination examples of indexes for risk assessment.

FIG. 12 illustrates an example of risk assessment that is performed by a risk assessment unit according to the fourth exemplary embodiment.

FIG. 13 is a flowchart illustrating a procedure in which a radiation generation apparatus changes a content of a notification to notify the user that a communication state is unsuitable for image capturing using the automatic exposure control function, in a radiation imaging system according to the fourth exemplary embodiment.

### DESCRIPTION OF THE EMBODIMENTS

Exemplary embodiments of the present disclosure will be described below with reference to the accompanying drawings. Details of the configuration of each exemplary embodiment are not limited to the descriptions and drawings therein. According to the present specification, a radiation includes not only X-ray but also α-ray, β-ray, γ-ray, corpuscular ray, and cosmic ray.

### System Configuration

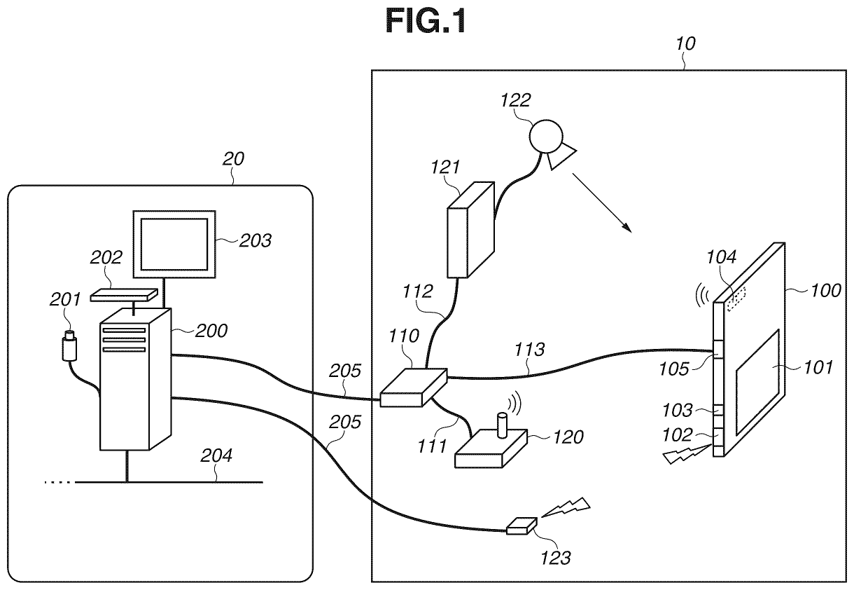

FIG. 1 illustrates a configuration example of a radiation imaging system according to a first exemplary embodiment.

As illustrated in FIG. 1, the radiation imaging system includes a radiation room 10 in which radiation imaging with radiation irradiation is performed, and a control room 20 disposed in the vicinity of the radiation room 10.

The radiation room 10 includes a radiation imaging apparatus 100, a communication control apparatus 110, an access point 120, a radiation generation apparatus 121, a radiation source 122, and an entry apparatus 123. The radiation room 10 further includes an access point (AP) communication cable 111, a radiation generation apparatus communication cable 112, and a sensor communication cable 113.

The control room 20 includes an information processing apparatus 200, a radiation irradiation switch 201, an input apparatus 202, a display apparatus 203, an in-hospital local area network (LAN) 204, and radiation room communication cables 205.

The radiation imaging apparatus 100 includes a power source control unit 101 including a battery, a short distance wireless communication unit 102, a switch 103, a wireless communication unit 104, and a wired communication unit 105. The radiation imaging apparatus 100 detects radiation that has been emitted from the radiation source 122 of the radiation generation apparatus 121 and penetrated a subject (not illustrated), and generates radiation image data.

The access point 120 is used in wireless communication between the radiation imaging apparatus 100 and the radiation generation apparatus 121 and between the radiation imaging apparatus 100 and the information processing apparatus 200 via the communication control apparatus 110. Wired communication using the sensor communication cable 113 is also usable between the radiation imaging apparatus 100 and the communication control apparatus 110. In the present exemplary embodiment, as an example, wireless communication using the access point 120 is used.

The radiation generation apparatus 121 controls the radiation source 122 to irradiate a subject with radiation (the arrow in FIG. 1). The radiation generation apparatus 121 includes a radiation source control unit that controls the radiation source 122 to perform the radiation irradiation based on a predetermined condition, and a radiation generation control unit that controls radiation generation in accordance with a radiation start/stop signal from the radiation imaging apparatus 100. The radiation source control unit and the radiation generation control unit may be separate apparatuses.

The AP communication cable 111 connects the access point 120 and the communication control apparatus 110. The radiation generation apparatus communication cable 112 connects the radiation generation apparatus 121 and the communication control apparatus 110.

The information processing apparatus 200 communicates with the radiation imaging apparatus 100 and the radiation generation apparatus 121 via the communication control apparatus 110 to control the entire operation of the radiation imaging system.

The radiation irradiation switch 201 is used by a user (not illustrated) to input a radiation irradiation timing. The input apparatus 202 receives an input of an instruction from the user and includes a keyboard, a touch panel, and other various input devices. The display apparatus 203 as a display unit displays radiation image data having been subjected to image processing and a graphical user interface (GUI). The in-hospital LAN 204 is an in-house backbone network. The radiation room communication cables 205 connect the information processing apparatus 200 in the control room 20 with the communication control apparatus 110 and the entry apparatus 123 in the radiation room 10.

Operations of the radiation imaging system (radiation detection system) will be described below.

The user performs an operation for registering the radiation imaging apparatus 100 (radiation detection apparatus) to the radiation imaging system. In response to the user pressing the switch 103 of the radiation imaging apparatus 100, short distance wireless communication is started between the short distance wireless communication unit 102 of the radiation imaging apparatus 100 and the entry apparatus 123.

The information processing apparatus 200 transmits wireless connection information on the access point 120 to the radiation imaging apparatus 100 via short distance wireless communication of the entry apparatus 123. For example, the wireless connection information on a wireless LAN includes information of a communication method such as IEEE 802.11, information of a physical channel, a service set identifier (SSID), and an encryption key.

The radiation imaging apparatus 100 sets the wireless communication unit 104 according to the received wireless LAN connection information. In accordance with the setting, the radiation imaging apparatus 100 establishes a wireless communication connection with the access point 120.

Then, the user inputs subject information, such as the identifier (ID), name, and birth date of the subject, and an imaging portion of the subject. After inputting of an imaging portion, the user fixes the subject's posture and the position of the radiation imaging apparatus 100.

After completion of imaging preparation, the user presses the radiation irradiation switch 201. In response to the radiation irradiation switch 201 being pressed, the radiation source 122 irradiates the subject with a radiation.

The radiation imaging apparatus 100 wirelessly communicates with the radiation generation apparatus 121 to control the start and stop of the radiation irradiation. The radiation emitted to the subject penetrates the subject and is incident on the radiation imaging apparatus 100. After a conversion of the incident radiation into visible light, the radiation imaging apparatus 100 detects the light as a radiation image signal by using a photoelectric conversion element as a detector.

The radiation imaging apparatus 100 drives the photoelectric conversion element to read the radiation image signal, and an analog-to-digital (A/D) conversion circuit converts the analog signal into a digital signal to obtain radiation image data. The obtained radiation image information (radiation image data) is transferred from the radiation imaging apparatus 100 to the information processing apparatus 200 via wireless communication.

The information processing apparatus 200 performs image processing on the received radiation image data. The information processing apparatus 200 displays, on the display apparatus 203, a radiation image based on the radiation image data having been subjected to the image processing.

The information processing apparatus 200 functions as an image processing apparatus and a display control apparatus.

### Hardware Configuration of Radiation Imaging Apparatus 100

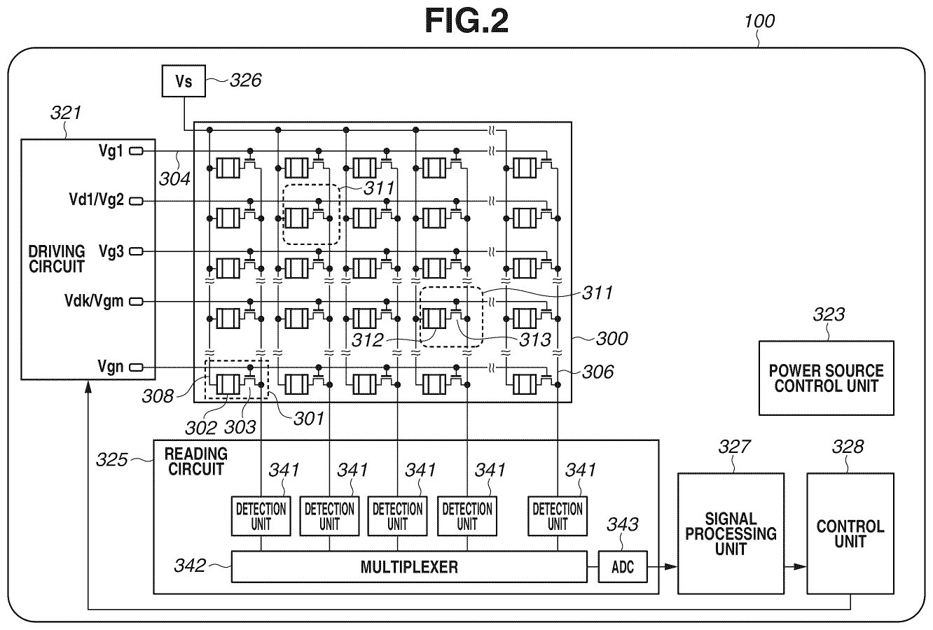

FIG. 2 illustrates a hardware configuration example of the radiation imaging apparatus 100.

As illustrated in FIG. 2, the radiation imaging apparatus 100 includes a radiation detector 300 as a detection unit for detecting a radiation. The radiation detector 300 has a function of detecting the emitted radiation. The radiation detector 300 includes a plurality of pixels arranged in rows and columns. In the following descriptions, a region where the plurality of pixels is arranged in the radiation detector 300 is referred to as an imaging region. The plurality of pixels includes a plurality of imaging pixels 301 for acquiring radiation image data and a plurality of detection pixels 311 for monitoring radiation irradiation. The detection pixels 311 are used for automatic exposure control.

An imaging pixel 301 includes a first conversion element 302 for converting a radiation into an electrical signal, and a first switch 303 disposed between a column signal line 306 and the first conversion element 302.

A detection pixel 311 includes a second conversion element 312 for converting a radiation into an electrical signal, and a second switch 313 disposed between the column signal line 306 and the second conversion element 312. The detection pixel 311 is disposed in the same column as some of the plurality of the imaging pixels 301.

The first conversion element 302 and the second conversion element 312 each include a scintillator for converting a radiation into light, and the photoelectric conversion element for converting light into an electrical signal. The scintillator is generally formed in sheet form to cover the imaging region and shared by a plurality of pixels. Alternatively, the first conversion element 302 and the second conversion element 312 each include a conversion element for directly converting a radiation into light.

For example, the first switch 303 and the second switch 313 each include a thin film transistor (TFT) having an activation region made of a semiconductor, such as amorphous silicon or polycrystalline silicon (preferably polycrystalline silicon).

The radiation imaging apparatus 100 includes a plurality of column signal lines 306 and a plurality of drive lines 304.

Each column signal line 306 corresponds to a different one of the plurality of columns in the imaging region. The drive lines 304 each correspond to a different one of the plurality of rows in the imaging region. A plurality of the drive lines 304 is driven by a driving circuit 321.

A first electrode of the first conversion element 302 is connected to a first main electrode of the first switch 303, and a second electrode of the first conversion element 302 is connected to a bias line 308. Each bias line 308 is commonly connected to the second electrodes of a plurality of the first conversion elements 302 extending and being arranged in the column direction.

The bias line 308 is supplied with a bias voltage Vs supplied from an element power source circuit 326. A power source control unit 323 includes a battery and a direct-current to direct-current (DC/DC) converter.

Second main electrodes of the first switches 303 of the plurality of the imaging pixels 301 included in one column are connected to one column signal line 306. Control electrodes of the first switches 303 of the plurality of the imaging pixels 301 included in one row are connected to one drive line 304. The plurality of the column signal lines 306 is connected to a reading circuit 325. The reading circuit 325 includes a plurality of detection units 341, a multiplexer 342, and an analog-to-digital converter (hereinafter referred to as an A/D converter) 343.

Each of the plurality of the column signal lines 306 is connected to a corresponding one of the plurality of the detection units 341 in the reading circuit 325. One column signal line 306 corresponds to one detection unit 341. A detection unit 341 includes, for example, a differential amplifier. The multiplexer 342 selects the plurality of the detection units 341 in a predetermined order, and supplies a signal from the selected detection unit 341 to the A/D converter 343. The A/D converter 343 converts the supplied signal into a digital signal and outputs the digital signal. The output of the reading circuit 325 (A/D converter 343) is supplied to a signal processing unit 327 and then processed by the signal processing unit 327. The signal processing unit 327 outputs information indicating the radiation irradiation to the radiation imaging apparatus 100, based on the output of the reading circuit 325 (A/D converter 343).

The connection form of the second conversion element 312 of the detection pixel 311 is similar to that of the imaging pixel 301. In driving the detection pixels 311, the driving circuit 321 drives the detection pixels 311 via each drive line 304. When the detection pixels 311 are driven, the signal processing unit 327 outputs information indicating the radiation irradiation to the radiation imaging apparatus 100, based on the output of the reading circuit 325 (A/D converter 343). More specifically, for example, the signal processing unit 327 detects the radiation irradiation to the radiation imaging apparatus 100 and calculates the radiation dose and/or the cumulative dose. The calculated radiation dose and cumulative dose may be transmitted to the information processing apparatus 200 and the radiation generation apparatus 121 via a communication unit 402 (described below). In this case, the communication unit 402 functions as a communication unit for transmitting radiation information.

The detection pixels 311 may have the same structure as the imaging pixels 301.

A control unit 328 controls the driving circuit 321 and the reading circuit 325, based on information from the signal processing unit 327 and control commands from the information processing apparatus 200.

### Configuration of Control Unit 328 of Radiation Imaging Apparatus 100

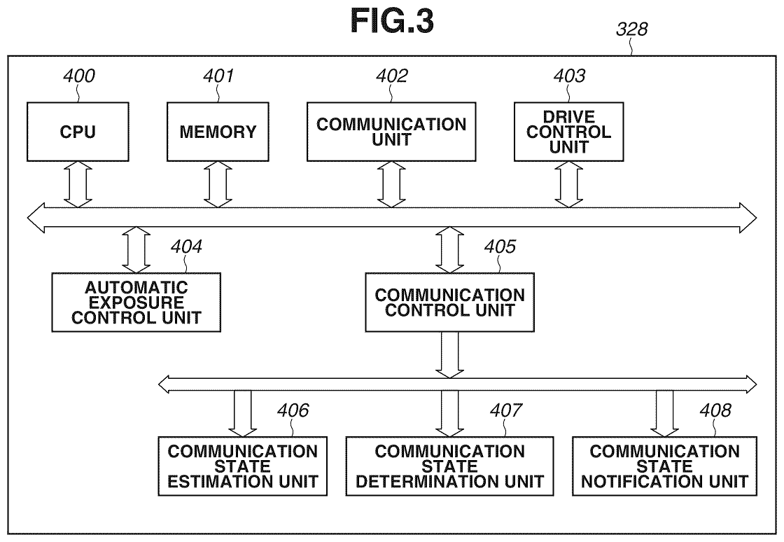

FIG. 3 illustrates a configuration of the control unit 328 of the radiation imaging apparatus 100 according to the first exemplary embodiment.

As illustrated in FIG. 3, the control unit 328 of the radiation imaging apparatus 100 includes a central processing unit (CPU) 400, a memory 401, the communication unit 402, a drive control unit 403, an automatic exposure control unit 404, a communication control unit 405, a communication state estimation unit 406, a communication state determination unit 407, and a communication state notification unit 408.

The CPU 400 uses the memory 401 as a work memory and executes various programs stored in a storage medium of a program storage unit to control the entire operation of the radiation imaging apparatus 100. As described in detail below, the CPU 400 controls whether to perform the automatic exposure control to detect radiation during the radiation irradiation. The memory 401 stores, reads, and writes therein various data to be processed by the CPU 400. The communication unit 402 is controlled by the CPU 400 via the communication control unit 405 and communicates with the radiation generation apparatus 121 and the information processing apparatus 200 via the communication control apparatus 110 by using the wireless communication unit 104 or the wired communication unit 105. The drive control unit 403 is controlled by the CPU 400 and controls the driving circuit 321 and the reading circuit 325, in accordance with information from the signal processing unit 327 and commands from the information processing apparatus 200.

The automatic exposure control unit 404 is controlled by the CPU 400 and performs a dose control operation of the automatic exposure control function. More specifically, the automatic exposure control unit 404 detects radiation incident on an interest region with the detection pixels 311 and calculates a cumulative dose as the total value of a detected radiation dose. More specifically, the CPU 400 performs a radiation detection operation during radiation irradiation. Then, the automatic exposure control unit 404 determines whether a radiation irradiation stop condition of the automatic exposure control function is satisfied, based on the calculated cumulative dose. In a case where the automatic exposure control unit 404 determines that the radiation irradiation stop condition is satisfied, the automatic exposure control unit 404 issues a radiation irradiation stop notification to the radiation generation apparatus 121 via the communication unit 402. More specifically, the automatic exposure control unit 404 functions as a control unit that performs the radiation stop control in association with the communication unit 402. Then, the radiation generation apparatus 121 stops the radiation irradiation, based on the notified radiation irradiation stop timing. While the radiation imaging apparatus 100 issues a radiation irradiation stop notification to the radiation generation apparatus 121 as a result of the radiation detection, the present disclosure is not limited thereto. The radiation imaging apparatus 100 may transmit the cumulative dose as a detection result at predetermined time intervals to the information processing apparatus 200 via the communication unit 402. The information processing apparatus 200 may calculate the cumulative doses and issues a radiation irradiation stop notification to the radiation generation apparatus 121. In this case, the information processing apparatus 200 functions as a control unit that performs the irradiation radiation stop control. Alternatively, the radiation imaging apparatus 100 may transmit a cumulative dose as a detection result at predetermined time intervals to the radiation generation apparatus 121 via the communication unit 402. The radiation generation apparatus 121 may calculate a total value of the cumulative doses. The irradiation radiation stop timing may be determined based on a maximum irradiation time period that has been input to the information processing apparatus 200 by the user.

The communication control unit 405 is controlled by the CPU 400 and communicates with the radiation generation apparatus 121 and the information processing apparatus 200 via the communication control apparatus 110 via the communication unit 402.

The communication state estimation unit 406 is controlled by the communication control unit 405 and estimates a communication state of communication, for example, between the radiation imaging apparatus 100 and the communication control apparatus 110 and between the radiation imaging apparatus 100 and radiation generation apparatus 121, in the radiation imaging system. In the communication state estimation, index data representing a communication state is used. Examples of the index data include a time taken for packet transmission and reception, a retransmission frequency, a carrier sense occurrence frequency, and received signal strength indication (RSSI). The present disclosure is not limited thereto, and any other indexes are also applicable as long as the indexes relate to the occurrence of a communication delay.

The communication state determination unit 407 is controlled by the communication control unit 405 and determines whether the communication state estimated by the communication state estimation unit 406 is suitable for image capturing using the automatic exposure control function. More specifically, the communication state determination unit 407 manages a threshold value that is compared with the index data used for the communication state estimation by the communication state estimation unit 406 to classify the communication state as a communication state suitable for image capturing using the automatic exposure control function or a communication state unsuitable for image capturing using the automatic exposure control function. More specifically, the communication state determination unit 407 manages the threshold value corresponding to the index that is used by the communication state estimation unit 406. For example, in a case where the communication state estimation unit 406 estimates a communication state by using a retransmission frequency (e.g., retransmission frequency in packet transmission) in the information transmission as an index, the communication state determination unit 407 determines the communication state based on a comparison between a threshold value of the retransmission frequency and the retransmission frequency. In a case where the communication state estimation unit 406 estimates the communication state by using a time period taken for information communication by the communication unit 402, i.e., a communication time period, as an index, the communication state determination unit 407 determines the communication state by using a threshold value of the communication time period. Then, the communication state determination unit 407 determines the communication state exceeding the threshold value as a defective communication, and determines that the communication state is unsuitable for image capturing using the automatic exposure control function. The threshold value that is used by the communication state determination unit 407 may be appropriately changeable by an instruction from an apparatus in the radiation imaging system, such as the information processing apparatus 200.

In a case where the communication state determination unit 407 determines that the communication state is unsuitable for image capturing using the automatic exposure control function, the communication state notification unit 408 which is controlled by the communication control unit 405 notifies the user that the communication state is unsuitable for image capturing using the automatic exposure control function, as a determination result. More specifically, the communication state notification unit 408 notifies the information processing apparatus 200 of the determination result via the communication unit 402. The information processing apparatus 200 notifies the user of a message indicating that the communication state is unsuitable for image capturing using the automatic exposure control function, as warning information for the stop control via the display apparatus 203.

### Flowchart of Procedure to Notify Communication State Unsuitable for Image Capturing Using the Automatic Exposure Control Function

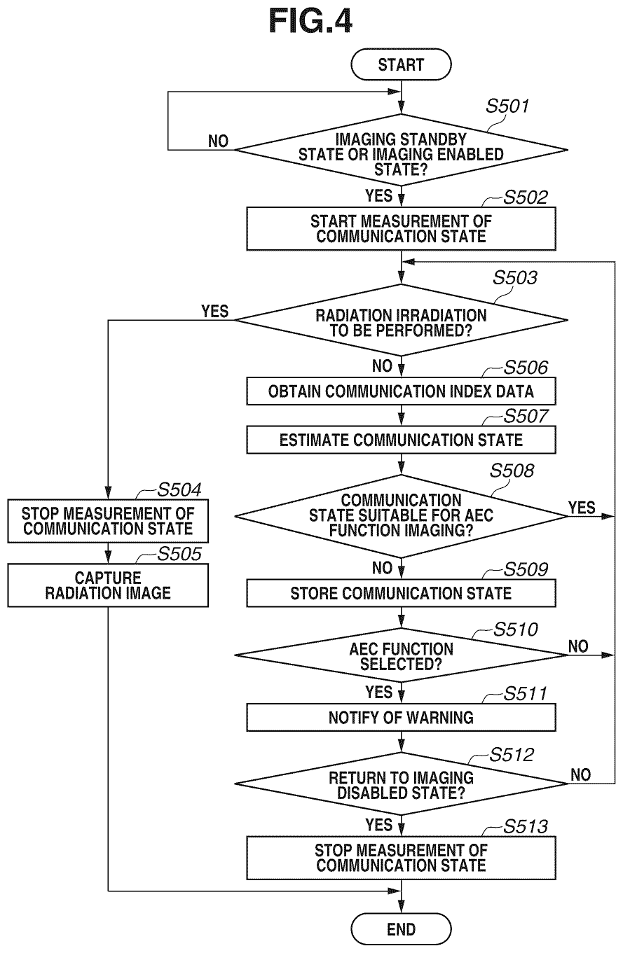

FIG. 4 is a flowchart illustrating a procedure in which the radiation imaging apparatus 100 notifies the user of the communication state unsuitable for image capturing using the automatic exposure control function, in the radiation imaging system according to the present exemplary embodiment.

In step S501, the drive control unit 403 of the radiation imaging apparatus 100 determines whether the radiation imaging apparatus 100 is in an imaging standby state during a transition from an imaging disabled state to an imaging enabled state or in the imaging enabled state after the radiation imaging apparatus 100 has been received radiation irradiation. In a case where the radiation imaging apparatus 100 determines that the communication state is either of the states (YES in step S501), the processing proceeds to step S502. In a case where the radiation imaging apparatus 100 determines that the communication state is neither of the states (NO in step S501), the processing returns to step S501. The imaging disabled state of the radiation imaging apparatus 100 refers to a state where image generation is not to be performed even if the radiation imaging apparatus 100 receives radiation irradiation, or a state where the power consumption is able to be reduced in comparison with the imaging enabled state.

In step S502, the communication control unit 405 of the radiation imaging apparatus 100 starts measurement of the communication state by using the communication state estimation unit 406.

In step S503, the drive control unit 403 of the radiation imaging apparatus 100 determines whether the user has pressed the radiation irradiation switch 201 to issue a radiation irradiation request to the radiation generation apparatus 121. In a case where the radiation imaging apparatus 100 determines that a radiation irradiation request has not been issued and radiation irradiation is not to be performed (NO in step S503), the processing proceeds to step S506. In a case where the radiation imaging apparatus 100 determines that a radiation irradiation request has been issued and radiation irradiation is to be performed (YES in step S503), the processing proceeds to step S504.

In step S504, to prevent occurrence of a communication noise in a radiation image, the communication control unit 405 of the radiation imaging apparatus 100 stops the measurement of the communication state using the communication state estimation unit 406.

In step S505, the drive control unit 403 and the automatic exposure control unit 404 of the radiation imaging apparatus 100 generate a radiation image in response to receipt of radiation emitted from the radiation generation apparatus 121, and perform the automatic exposure control.

In step S506, the communication state estimation unit 406 of the radiation imaging apparatus 100 obtains communication index data as a result of the measurement of the communication state started in step S502. As described above, the obtained communication index data is an index that is related to occurrence of a communication delay. While examples of communication index data include a time period taken for packet transmission and reception, a retransmission frequency, a carrier sense occurrence frequency, and RSSI, the present disclosure is not limited thereto.

In step S507, the communication state estimation unit 406 of the radiation imaging apparatus 100 estimates the communication state in the radiation imaging system, based on the obtained communication index data. While examples of methods for estimating the communication state include a method for using a statistical value of the obtained communication index data and a method for using a retransmission frequency and a carrier sense occurrence frequency, the present invention is not limited thereto. Any other methods are also applicable as long as the methods define the event probability of a communication delay.

In step S508, the communication state determination unit 407 of the radiation imaging apparatus 100 determines whether the communication state is suitable for image capturing using the automatic exposure control function, based on the communication state estimated in step S507. In a case where the communication state determination unit 407 determines that the communication state is unsuitable for image capturing using the automatic exposure control function (NO in step S508), the processing proceeds to step S509. In a case where the communication state determination unit 407 determines that the communication state is suitable for image capturing using the automatic exposure control function (YES in step S508), the processing returns to step S503. In a case where the communication state has been estimated based on the statistical value of the communication time period in step S507, the communication state determination unit 407 may determine the communication state in step S508 by comparing the statistical value with the threshold value. Alternatively, for example, the communication state estimation unit 406 may estimate the communication time period at a plurality of times, and the communication state determination unit 407 may determine the communication state based on the number of times of the estimated communication time period exceeding a set threshold value.

In step S509, the communication state determination unit 407 of the radiation imaging apparatus 100 stores the communication index data based on which the communication state has been determined to be unsuitable for image capturing using the automatic exposure control function.

In step S510, the communication state notification unit 408 of the radiation imaging apparatus 100 checks whether image capturing using the automatic exposure control function is selected in the current image capturing setting of the radiation imaging system. In a case where image capturing using the automatic exposure control function is selected (YES in step S510), the processing proceeds to step S511. In a case where image capturing using the automatic exposure control function is not selected (NO in step S510), the processing returns to step S503.

In step S511, the communication state notification unit 408 of the radiation imaging apparatus 100 notifies the information processing apparatus 200 that the communication state is unsuitable for image capturing using the automatic exposure control function, via the communication unit 402. The information processing apparatus 200 notifies the user that the communication state is unsuitable for image capturing using the automatic exposure control function, via the display apparatus 203.

In step S512, the drive control unit 403 of the radiation imaging apparatus 100 determines whether the radiation imaging apparatus 100 is to be shifted from the imaging enabled state, in which the radiation imaging apparatus 100 is ready to perform image capturing in response to receipt of radiation irradiation, to the imaging disabled state (non-test state described below). In a case where the drive control unit 403 determines to shift the radiation imaging apparatus 100 to the imaging disabled state (YES in step S512), the processing proceeds to step S513. In a case where the drive control unit 403 determines not to shift the radiation imaging apparatus 100 to the imaging disabled state (NO in step S512), the processing returns to step S503.

In step S513, to shift the radiation imaging apparatus 100 back to the imaging disabled state, the communication control unit 405 of the radiation imaging apparatus 100 stops the measurement of the communication state using the communication state estimation unit 406.

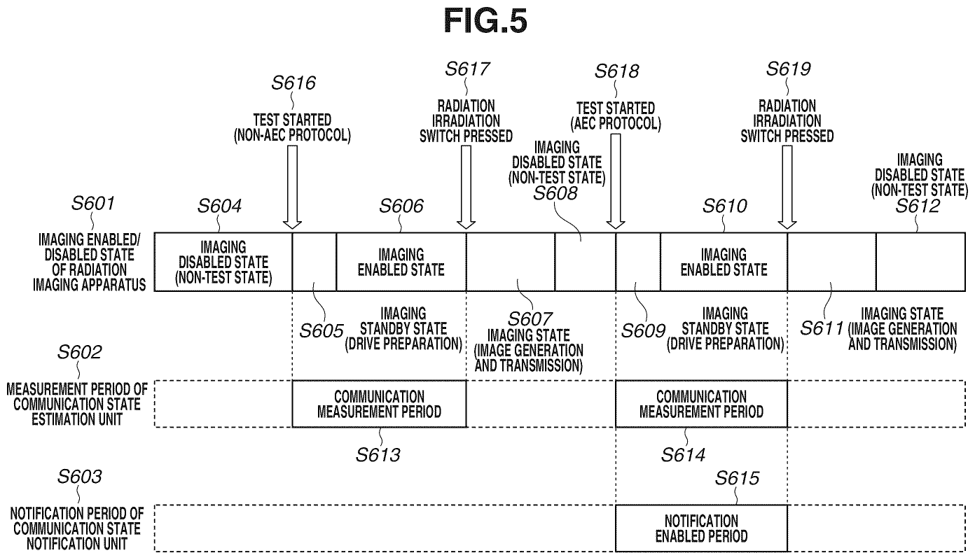

FIG. 5 illustrates a relation between the imaging enabled/disabled state of the radiation imaging apparatus 100, a communication measurement period of the communication state estimation unit 406, and a user notification enabled period of the communication state notification unit 408, in the radiation imaging system according to the present exemplary embodiment. FIG. 5 illustrates an example case in which imaging is performed once without using the automatic exposure control function and then imaging is performed once using the automatic exposure control function.

Referring to FIG. 5, changes in the communication measurement period of the communication state estimation unit 406 and the user notification enabled period of the communication state notification unit 408 over time will be described below in association with the flowchart in FIG. 4.

Step S601 indicates a state transition in the imaging enabled/disabled state of the radiation imaging apparatus 100 of the radiation imaging system with reference to steps S604 to S612. The drawing indicates the passage of time from left to right. As illustrated in blocks, the radiation imaging apparatus 100 has four different states: the imaging disabled state (non-test state), the imaging standby state, the imaging enabled state, and an imaging state. Step S602 indicates a state transition in the communication measurement period of the communication state estimation unit 406 with reference to steps S613 and S614. Step S603 indicates a state transition in the user notification enabled period of the communication state notification unit 408 with reference to step S615. Steps S616 and S618 each indicate a timing when a radiation imaging test is started. Steps S617 and S619 each indicate a timing when the user presses the radiation irradiation switch 201.

In step S616, the user operates the information processing apparatus 200 to start an imaging test not using the automatic exposure control function. In this processing, the radiation imaging apparatus 100 enters, from the imaging disabled state (non-test state) in step S604, to the imaging standby state (a drive preparation period) in step S605 and the imaging enabled state in step S606 in this order. The imaging standby state in step S605 is a part of the imaging disabled state in which the radiation irradiation by the radiation generation apparatus 121 is not permitted in the radiation imaging system. The radiation irradiation by the radiation generation apparatus 121 is permitted at a timing when the radiation imaging apparatus 100 enters the imaging enabled state in step S606.

As illustrated in step S613, the communication state estimation unit 406 starts the measurement of the communication state at the timing when the user operates the information processing apparatus 200 to start image capturing not using the automatic exposure control function. More specifically, the measurement of the communication state is started before the radiation irradiation. This timing is the same as the timing when the radiation imaging apparatus 100 enters the imaging standby state (drive preparation period) in step S605 from the imaging disabled state (non-test state) in step S604. The above descriptions correspond to the processing of steps S501 (YES) and S502 in the flowchart in FIG. 4. In steps S613 and S614, the communication state estimation unit 406 performs the measurement of the communication state with a plurality of transmission and reception sequences in the communication measurement period.

Even in a case where the communication state determination unit 407 determines that the communication state is unsuitable for image capturing using the automatic exposure control function in the communication measurement period in step S613, the communication state notification unit 408 does not notify the user that the communication state is unsuitable for image capturing using the automatic exposure control function, as illustrated in step S603. The communication measurement period in step S613 is the period of the imaging test not using the automatic exposure control function started in step S616. Thus, in this period, the user does not need a notification about the communication state unsuitable for image capturing using the automatic exposure control function. The above descriptions correspond to the processing of steps S503 (NO), S506, S507, S508 (NO), S509, and S510 (NO) in the flowchart in FIG. 4.

In step S617, in response to the user pressing the radiation irradiation switch 201, the radiation imaging system starts radiation image capturing not using the automatic exposure control function. In this processing, the radiation imaging apparatus 100 enters the imaging state in step S607 from the imaging enabled state in step S606 to perform image generation and transmission. At the same timing as this transition, the communication state estimation unit 406 stops the measurement of the communication state. After completion of the image generation and transmission, the radiation imaging apparatus 100 enters the non-test state in step S608 from the imaging state in step S607. The above descriptions correspond to the processing of steps S503 (YES), S504, and S505 in the flowchart in FIG. 4. While, in the example illustrated in FIG. 5, the radiation imaging apparatus 100 enters the imaging disabled state (non-test state) in step S608 in response to switching of the imaging test, the present disclosure is not limited thereto. After completion of the image generation and transmission, the radiation imaging apparatus 100 may enter the imaging standby state in step S609 for the next image capturing from the imaging state in step S607.

In step S618, the user operates the information processing apparatus 200 to start the imaging test using the automatic exposure control function. In this processing, the radiation imaging apparatus 100 enters the imaging standby state (drive preparation period) in step S609 from the imaging disabled state (non-test state) in step S608 and then enters the imaging enabled state in step S610.

As illustrated in step S614, the communication state estimation unit 406 starts the measurement of the communication state from the timing when the user operates the information processing apparatus 200 to start to set the image capturing setting using the automatic exposure control function. At the same timing, as illustrated in step S615, the communication state notification unit 408 also starts a period in which the user is notified of the communication state unsuitable for image capturing using the automatic exposure control function. This timing is the same as the timing when the radiation imaging apparatus 100 enters the imaging standby state (drive preparation period) in step S609 from the imaging disabled state (non-test state) in step S608. The above descriptions correspond to the processing of steps S501 (YES) and S502 in the flowchart in FIG. 4.

When the communication state determination unit 407 determines that the communication state is unsuitable for image capturing using the automatic exposure control function in the communication measurement period in step S614, the communication state notification unit 408 notifies the user that the communication state is unsuitable for image capturing using the automatic exposure control function. The above descriptions correspond to the processing of steps S503 (NO), S506, S507, S508 (NO), S509, S510 (YES), S511, and S512 (NO) in the flowchart in FIG. 4.

In step S619, in response to the user pressing the radiation irradiation switch 201, the radiation imaging system starts radiation image capturing using the automatic exposure control function. In this processing, the radiation imaging apparatus 100 enters the imaging state in step S611 from the imaging enabled state in step S610 to perform the image generation and transmission. At the same timing as this transition, the communication state estimation unit 406 stops the measurement of the communication state, and the communication state notification unit 408 ends the period in which the user is notified of the communication state unsuitable for image capturing using the automatic exposure control function. After completion of the image generation and transmission, the radiation imaging apparatus 100 enters the imaging disabled state (non-test state) in step S612 from the imaging state in step S611. The above descriptions correspond to the processing of steps S503 (YES), S504, and S505 in the flowchart in FIG. 4.

As described above, the radiation imaging apparatus 100 of the radiation imaging system estimates the communication environment in the radiation imaging system by using the communication state estimation unit 406. In a case where the communication state determination unit 407 determines that the communication state is unsuitable for image capturing using the automatic exposure control function, the communication state notification unit 408 notifies the user that the communication state is unsuitable for image capturing using the automatic exposure control function. By receiving the state of the communication environment, the user is able to determine not to perform image capturing using the automatic exposure control function.

With this configuration, an increase in the dose of a patient due to a communication delay in the automatic exposure control is prevented, and generation of an image with a cumulative dose larger than the total cumulative dose set as a threshold value in image capturing using the automatic exposure control function is prevented. Further, with the control of start and end of the communication measurement period via the communication state estimation unit 406 in association with the imaging enabled/disabled state of the radiation imaging apparatus 100, the user is notified of the communication state unsuitable for image capturing using the automatic exposure control function in a case where the radiation imaging apparatus 100 is in the imaging enabled state. In a case where the radiation imaging apparatus 100 is in the imaging disabled state (non-test state), the power consumption of the radiation imaging apparatus 100 is able to be reduced by stopping the communication measurement via the communication state estimation unit 406. According to the present exemplary embodiment, in a case where the determination result is “NO” in step S512 in FIG. 4, the radiation imaging apparatus 100 repetitively issues a warning notification to notify that the communication state remains unchanged (when step S511 is repeated in the communication state unsuitable for image capturing using the automatic exposure control function). To avoid repeated issuance of warnings, the radiation imaging apparatus 100 may issue the warning information only in response to a change in the result of the communication state determination from a favorable communication to a defective communication.

According to the present exemplary embodiment, the communication state estimation unit 406, the communication state determination unit 407, and the communication state notification unit 408 of the radiation imaging apparatus 100 may be included in the radiation generation apparatus 121. The communication state determination unit 407 may manage a plurality of threshold values to be used for the communication state determination and perform a determination to classify the communication state as one of a plurality of states. Further, the communication state notification unit 408 may change a content of the notification according to the one of the plurality of communication states classified by the communication state determination unit 407. This classification will be described below with reference to a second exemplary embodiment.

According to the present exemplary embodiment, the communication state estimation unit 406, the communication state determination unit 407, and the communication state notification unit 408 of the radiation imaging apparatus 100 may be included in the communication control apparatus 110.

The communication state estimation unit 406 manages a plurality of communication measurement methods and changes the communication measurement method according to the imaging enabled/disabled state or the power consumption of the radiation imaging apparatus 100. This processing will be described below with reference to a third exemplary embodiment.

The description has been given of the present exemplary embodiment of the present disclosure.

Processing according to the second exemplary embodiment of the present disclosure will be described below. The redundant descriptions of drawings and elements according to the present exemplary embodiment similar to those according to the first exemplary embodiment will be omitted.

### System Configuration

A radiation imaging system configuration of the present exemplary embodiment is similar to that illustrated in FIG. 1.

### Hardware Configuration of Radiation Imaging Apparatus 100

The hardware configuration of a radiation imaging apparatus 100 of the present exemplary embodiment is similar to that illustrated in FIG. 2.

### Configuration of Control Unit 328 of Radiation Imaging Apparatus 100

The configuration of a control unit 328 of the radiation imaging apparatus 100 of the present exemplary embodiment is similar to the configuration illustrated in FIG. 3 with the exclusion of the communication state estimation unit 406, the communication state determination unit 407, and the communication state notification unit 408.

### Configuration of Radiation Generation Apparatus 121

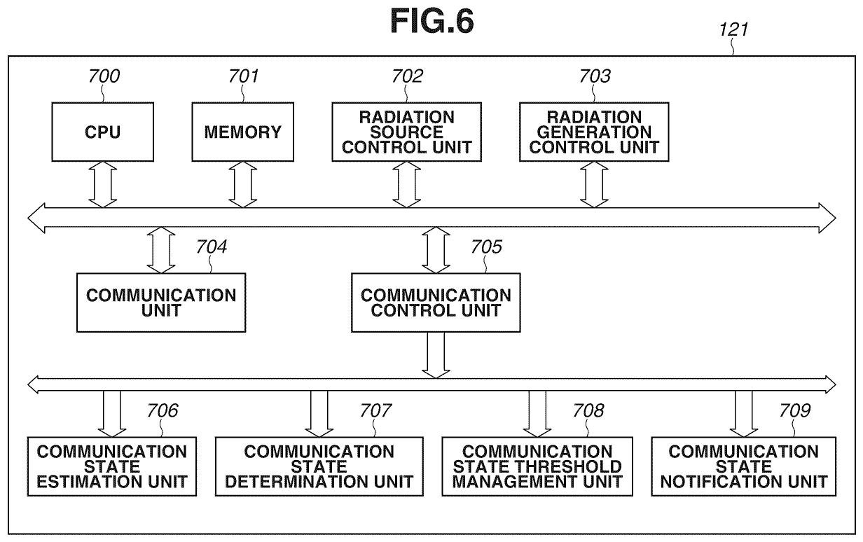

FIG. 6 illustrates a configuration of a radiation generation apparatus 121 of the radiation imaging system according to the present exemplary embodiment.

As illustrated in FIG. 6, the radiation generation apparatus 121 includes a CPU 700, a memory 701, a radiation source control unit 702, and a radiation generation control unit 703. The radiation generation apparatus 121 further includes a communication unit 704, a communication control unit 705, a communication state estimation unit 706, a communication state determination unit 707, a communication state threshold management unit 708, and a communication state notification unit 709.

The CPU 700 uses the memory 701 as a work memory and executes various programs stored in a storage medium of a program storage unit to control the entire operation of the radiation generation apparatus 121. The memory 701 stores, reads, and writes therein various data to be handled by the CPU 700. The radiation source control unit 702 is controlled by the CPU 700 and controls the radiation source 122 to perform radiation irradiation based on a predetermined condition. The radiation generation control unit 703 is controlled by the CPU 700 and controls radiation generation according to a radiation start or stop signal from the radiation imaging apparatus 100.

The communication unit 704 is controlled by the CPU 700 via the communication control unit 705 and communicates with the radiation imaging apparatus 100 and the information processing apparatus 200 via the communication control apparatus 110.

The communication state estimation unit 706 has a similar function to the communication state estimation unit 406. The communication state estimation unit 706 is controlled by the communication control unit 705 and estimates a communication state of communication, for example, between the radiation generation apparatus 121 and the radiation imaging apparatus 100 and between the radiation generation apparatus 121 and the communication control apparatus 110, in the radiation imaging system.

The communication state determination unit 707 has a similar function to the communication state determination unit 407. The communication state determination unit 707 is controlled by the communication control unit 705 and determines whether the communication state estimated by the communication state estimation unit 706 is suitable for image capturing using the automatic exposure control function. In this determination, the communication state determination unit 707 classifies the communication state estimated by the communication state estimation unit 706 as one of a plurality of states, based on a plurality of threshold values managed by the communication state threshold management unit 708.

The communication state notification unit 709 has a similar function to the communication state notification unit 408. The communication state notification unit 709 is controlled by the communication control unit 705 and notifies, in a case where the communication state determination unit 707 determines that the communication state is unsuitable for image capturing using the automatic exposure control function, the user that the communication state is unsuitable for image capturing using the automatic exposure control function. In this notification, the communication state notification unit 709 changes a content of the notification to the user in accordance with the one of the plurality of communication states determined by the communication state determination unit 707.

### Flowchart Indicating Procedure to Notify Communication State Unsuitable for Image Capturing Using Automatic Exposure Control Function

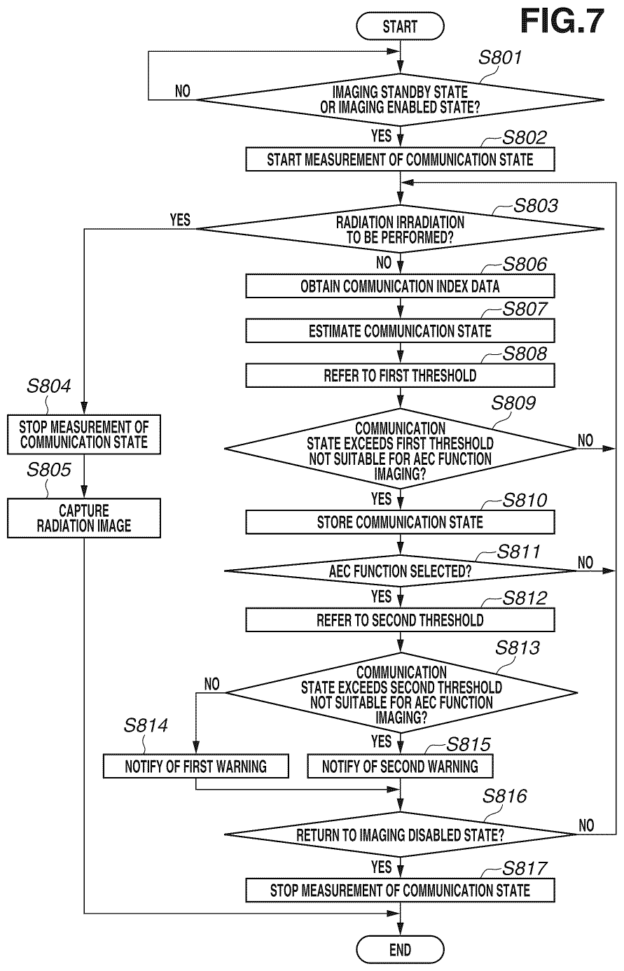

FIG. 7 is a flowchart illustrating a procedure in which the radiation generation apparatus 121 changes a content of the notification to the user according to a communication state, in the radiation imaging system according to the present exemplary embodiment. While, in this flowchart, a communication state is classified as one of three different states including a normal state, a warning state, and an error state in order of communication state normality, based on two different threshold values, the present disclosure is not limited thereto. Three or more threshold values may be used. As described above, the present exemplary embodiment uses a plurality of threshold values.

In step S801, the radiation generation apparatus 121 performs a determination similar to the determination in step S501. In step S801, the radiation generation apparatus 121 determines whether the radiation imaging apparatus 100 is in the imaging standby state during the transition from the imaging disabled state to the imaging enabled state or in the imaging enabled state, in which the radiation imaging apparatus 100 is ready to perform image capturing in response to receipt of radiation irradiation. In a case where the radiation generation apparatus 121 determines that the radiation imaging apparatus 100 is in either of the states (YES in step S801), the processing proceeds to step S802. In a case where the radiation generation apparatus 121 determines that the radiation imaging apparatus 100 is in neither of the states (NO in step S801), the processing returns to step S801.

Processing in step S802 is similar to the processing in step S502. The communication control unit 705 of the radiation generation apparatus 121 starts measurement of the communication state by using the communication state estimation unit 706.

In step S803, the radiation generation apparatus 121 performs a determination similar to step S503. In step S803, the radiation generation apparatus 121 determines whether a radiation irradiation request has been received. In a case where the radiation generation apparatus 121 determines that a radiation irradiation request has not been received and radiation irradiation is not to be performed (NO in step S803), the processing proceeds to step S806. In a case where the radiation generation apparatus 121 determines that a radiation irradiation request has been received and radiation irradiation is to be performed (YES in step S803), the processing proceeds to step S804.

In step S804, to prevent occurrence of a communication noise in the radiation image, the communication control unit 705 of the radiation generation apparatus 121 stops the measurement of the communication state using the communication state estimation unit 706.

Processing in steps S805 to S807 are similar to the processing in steps S505 to S507, respectively.

In step S808, the communication state determination unit 707 of the radiation generation apparatus 121 refers to a first threshold value managed by the communication state threshold management unit 708. The first threshold value is used to classify the communication state as the normal state or the other states (warning state or error state).

In step S809, the communication state determination unit 707 of the radiation generation apparatus 121 determines whether the communication state is unsuitable for image capturing using the automatic exposure control function (warning state or error state). In a case where the communication state determination unit 707 determines that the communication state exceeds the first threshold value, which means that the communication state is in the warning state or the error state, (YES in step S809), the processing proceeds to step S810. In a case where the communication state determination unit 707 determines that the communication state does not exceed the first threshold value, which means that the communication state is in the normal state (NO in step S809), the processing returns to step S803.

Processing in steps S810 and S811 are similar to the processing in steps S509 and S510, respectively.

In step S812, the communication state determination unit 707 of the radiation generation apparatus 121 refers to a second threshold value managed by the communication state threshold management unit 708. The second threshold value is used to classify the communication state as the warning state or the error state.

In step S813, the communication state determination unit 707 of the radiation generation apparatus 121 determines whether the communication state is in the warning state or in the error state. In a case where the communication state determination unit 707 determines that the communication state does not exceeds the second threshold value, which means that the communication state is in the warning state (NO in step S813), the processing proceeds to step S814. In a case where the communication state determination unit 707 determines that the communication state exceeds the second threshold value, which means that the communication state is in the error state (NO in step S813), the processing proceeds to step S815.

In step S814, the communication state notification unit 709 of the radiation generation apparatus 121 notifies the information processing apparatus 200 that the communication state is in the warning state in which the communication state exceeds the first threshold value. The information processing apparatus 200 notifies the user that the communication state is unsuitable for image capturing using the automatic exposure control function (first warning state), via the display apparatus 203.

In step S815, the communication state notification unit 709 of the radiation generation apparatus 121 notifies the information processing apparatus 200 that the communication state is in the error state in which the communication state exceeds the second threshold value. The information processing apparatus 200 notifies the user that the communication state is unsuitable for image capturing using the automatic exposure control function (second warning state, i.e., the error state in which it is desirable that image capturing using the automatic exposure control function should be avoided) via the display apparatus 203. According to the present exemplary embodiment, the information processing apparatus 200 changes the warning information to be notified according to the threshold values used to determine the communication state.

Processing in steps S816 and S817 are similar to the processing in steps S512 and S513, respectively.

As described above, the radiation generation apparatus 121 of the radiation imaging system estimates the communication environment in the radiation imaging system by using the communication state estimation unit 706. Then, the communication state determination unit 707 classifies the communication state as one of a plurality of states, based on a plurality of threshold values managed by the communication state threshold management unit 708. The communication state notification unit 709 changes the content of the communication state notification to the user in accordance with one of the plurality of communication states classified by the communication state determination unit 707. This enables the user to identify a communication environment degraded situation according to the content of the notification from the radiation imaging system, which leads to user's determination of either to cancel image capturing using the automatic exposure control function or to improve the communication environment. The radiation imaging system may not permit image capturing using the automatic exposure control function in the communication environment worst state (third warning state). This configuration prevents the dose of a patient from being increased due to a communication delay in the automatic exposure control, and prevents generation of an image with a cumulative dose larger than the total cumulative dose set as a threshold value in image capturing using the automatic exposure control function.

The description has been given of the present exemplary embodiment of the present disclosure.

The third exemplary embodiment of the present disclosure will be described below. The redundant descriptions of drawings and elements according to the third exemplary embodiment similar to those according to the first and the second exemplary embodiments will be omitted.

### System Configuration

The system configuration of the present exemplary embodiment is similar to that illustrated in FIG. 1.

### Hardware Configuration of Radiation Imaging Apparatus 100

The hardware configuration of a radiation imaging apparatus 100 of the present exemplary embodiment is similar to that illustrated in FIG. 2.

### Configuration of Control Unit 328 of Radiation Imaging Apparatus 100

The configuration of a control unit 328 of the radiation imaging apparatus 100 of the present exemplary embodiment is similar to the configuration illustrated in FIG. 3 with the exclusion of the communication state estimation unit 406, the communication state determination unit 407, and the communication state notification unit 408.

### Configuration of Communication Control Unit 110

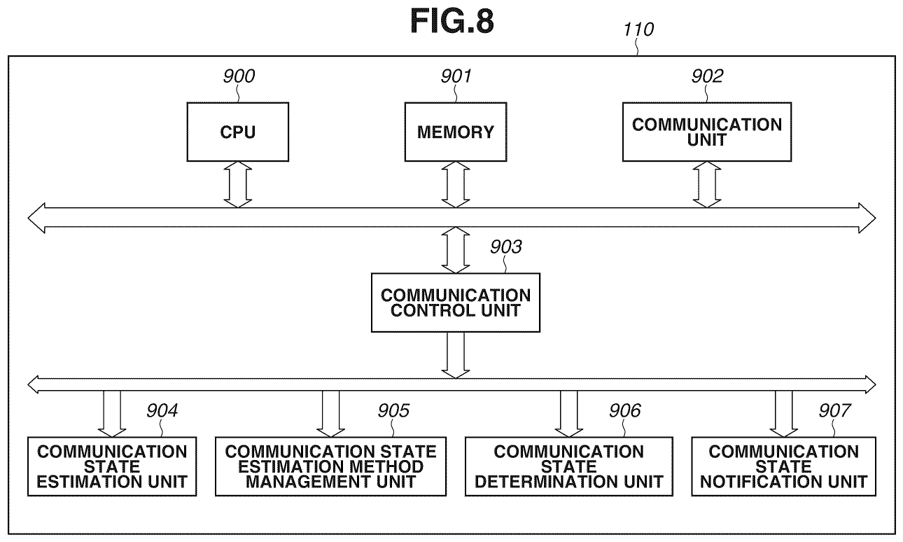

FIG. 8 illustrates a configuration of a communication control apparatus 110 of a radiation imaging system according to the present exemplary embodiment.

As illustrated in FIG. 8, the communication control apparatus 110 includes a CPU 900, a memory 901, a communication unit 902, a communication control unit 903, a communication state estimation unit 904, a communication state estimation method management unit 905, a communication state determination unit 906, and a communication state notification unit 907.

The CPU 900 uses the memory 901 as a work memory and executes various programs stored in a storage medium of a program storage unit to control the entire operation of the communication control apparatus 110. The memory 901 stores, reads, and writes therein various data to be processed by the CPU 900. The communication unit 902 is controlled by the CPU 900 via the communication control unit 903 and communicates with the radiation imaging apparatus 100, the radiation generation apparatus 121, and the information processing apparatus 200.

The communication state estimation unit 904 has a function similar to the communication state estimation unit 406. The communication state estimation unit 904 is controlled by the communication control unit 903. The communication state estimation unit 904 estimates a communication state of communication, for example, between the communication control apparatus 110 and the radiation imaging apparatus 100, between the communication control apparatus 110 and the radiation generation apparatus 121, and between the communication control apparatus 110 and the information processing apparatus 200, in the radiation imaging system.

The communication state estimation method management unit 905 manages a communication state estimation method to be used by the communication state estimation unit 904. The communication state estimation method management unit 905 manages algorithms and settings for estimating a communication state in the radiation imaging system in a case of managing a plurality of communication state estimation units each using a different algorithm and a case of changing a communication time interval in the same communication state estimation unit. The communication state estimation method management unit 905 is used in a case where the communication state estimation unit 904 changes the communication state estimation method.

The communication state determination unit 906 has a function similar to the communication state determination unit 407.

The communication state notification unit 907 has a function similar to the communication state notification unit 408.

### Flowchart Indicating Procedure to Notify Communication State Unsuitable for Image Capturing Using Automatic Exposure Control Function

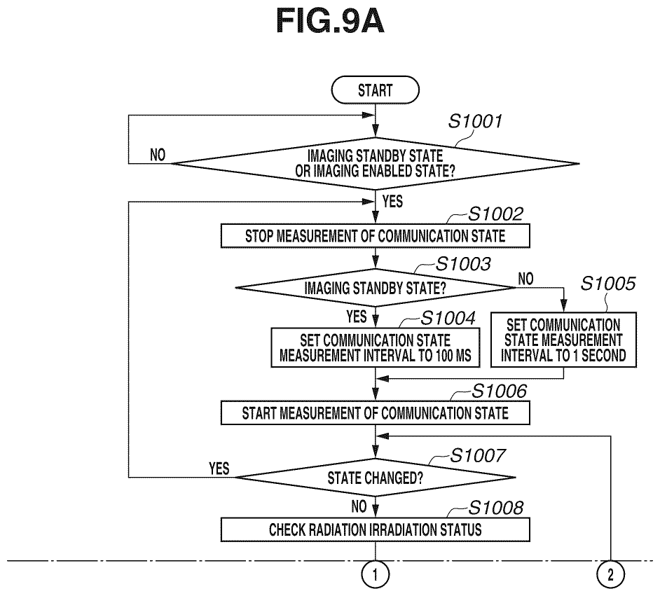

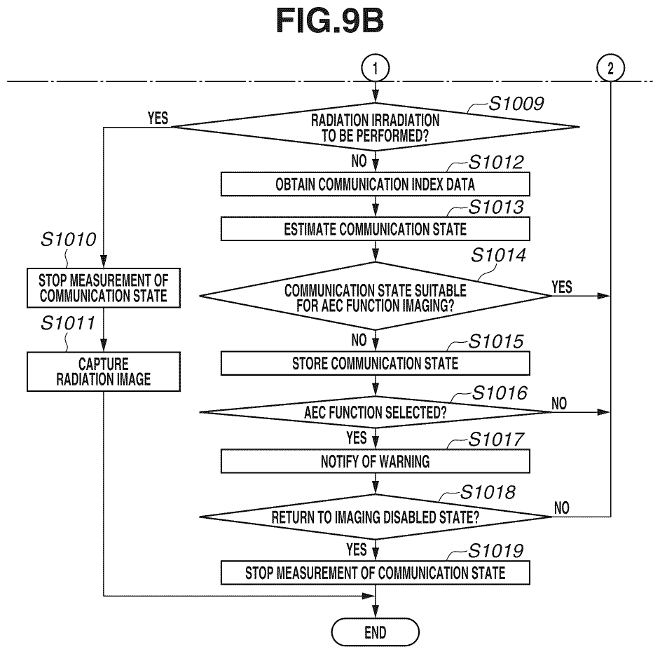

FIGS. 9A and 9B are a flowchart illustrating a procedure in which the communication control apparatus 110 notifies the user that the communication state is unsuitable for image capturing using the automatic exposure control function, in the radiation imaging system according to the present exemplary embodiment. While this flowchart indicates an example case in which an interval for measurement of a communication state is changed in the same communication state estimation unit, the present disclosure is not limited thereto, and the communication state estimation unit may be changed by selecting a communication state estimation unit using a different algorithm.

In step S1001, the communication control apparatus 110 performs a determination similar to the determination in step S501.

In step S1002, the communication control unit 903 of the communication control apparatus 110 stops the measurement of the communication state in response to the measurement being performed by using the communication state estimation unit 904.

In step S1003, the communication control apparatus 110 determines whether the radiation imaging apparatus 100 is in the imaging standby state during the transition from the imaging disabled state to the imaging enabled state. In a case where the communication control apparatus 110 determines that the radiation imaging apparatus 100 is in the imaging standby state (YES in step S1003), the processing proceeds to step S1004. In a case where the communication control apparatus 110 determines that the radiation imaging apparatus 100 is not in the imaging standby state and is in the imaging enabled state (NO in step S1003), the processing proceeds to step S1005.

In step S1004, the communication state estimation unit 904 of the communication control apparatus 110 uses the communication state estimation method management unit 905 to set the measurement interval of the communication state estimation unit 904 to 100 milliseconds. In step S1005, the communication state estimation unit 904 of the communication control apparatus 110 uses the communication state estimation method management unit 905 to set the measurement interval of the communication state estimation unit 904 to one second.

The processing in step S1006 is similar to the processing in step S502. In step S1006, the communication control apparatus 110 performs communication at the measurement intervals set in step S1004 or step S1005 during the measurement of the communication state.

In step S1007, the communication control apparatus 110 determines whether the radiation imaging apparatus 100 has entered the imaging enabled state from the imaging standby state or entered the imaging standby state from the imaging enabled state. In a case where the communication control apparatus 110 determines that the state of the radiation imaging apparatus 100 has not been changed (NO in step S1007), the processing proceeds to step S1008. In a case where the communication control apparatus 110 determines that the state of the radiation imaging apparatus 100 has been changed (YES in step S1007), the processing returns to step S1002.

In step S1008, the communication control apparatus 110 checks receipt of a radiation irradiation request issued to the radiation generation apparatus 121.

Processing in steps S1009 to S1019 are similar to the processing in steps S503 to S513, respectively.

As described above, the communication control apparatus 110 of the radiation imaging system uses the communication state estimation unit 904 to estimate the communication environment in the radiation imaging system. The communication state estimation unit 904 of the communication control apparatus 110 uses the communication state estimation method management unit 905 to change the communication state estimation method according to the state of the radiation imaging apparatus 100. Changing the communication state estimation method via the communication state estimation unit 904 allows the user to be appropriately notified of the communication state unsuitable for image capturing using the automatic exposure control function in a case where the radiation imaging apparatus 100 is in the imaging enabled state. Changing the communication state estimation method via the communication state estimation unit 904 also prevents an increase in the power consumption of the radiation imaging apparatus 100 due to the communication for the communication state estimation.

The description has been given of the present exemplary embodiment of the present disclosure.

While the first to third exemplary embodiments are based on a configuration in which the radiation imaging apparatus 100, the radiation generation apparatus 121, and the communication control apparatus 110 each include a communication state estimation unit, a communication state determination unit, and a communication state notification unit, the present invention is not limited thereto. For example, the information processing apparatus 200 may include a communication state estimation unit, a communication state determination unit, and a communication state notification unit. While the first to third exemplary embodiments are based on a configuration in which the user is notified of a result of the communication state determination (result of the comparison with a threshold value), the present disclosure is not limited thereto. For example, the user may be notified of only a result of the communication state estimation (result of the communication state detection), and a determination of suitability/unsuitability of the communication state may be left to the user. In this case, the determination unit (communication state determination unit) can be omitted, which is easier to implement the apparatus configuration (system configuration).

A fourth exemplary embodiment of the present disclosure will be described below. The redundant descriptions of drawings and elements according to the present exemplary embodiment similar to those according to the first, second, and third exemplary embodiments will be omitted.

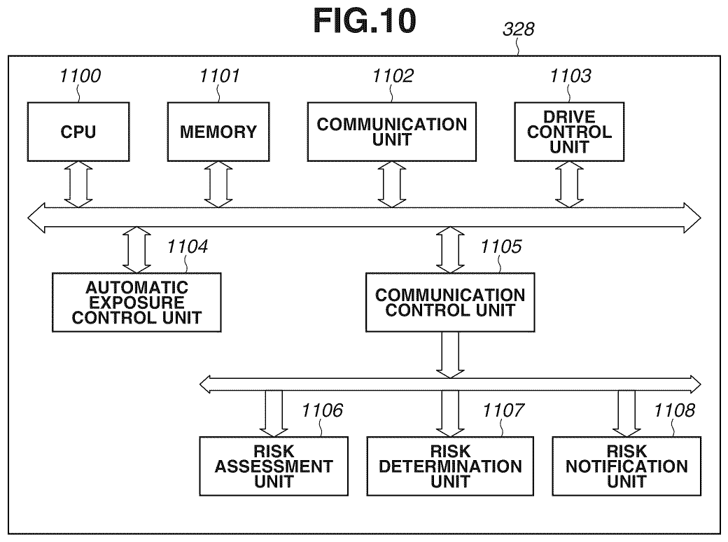

FIG. 10 illustrates a configuration of the control unit 328 of a radiation imaging apparatus 100 according to the fourth exemplary embodiment.

As illustrated in FIG. 10, the control unit 328 of the radiation imaging apparatus 100 includes a CPU 1100, a memory 1101, a communication unit 1102, a drive control unit 1103, an automatic exposure control unit 1104, a communication control unit 1105, a risk assessment unit 1106, a risk determination unit 1107, and a risk notification unit 1108.

The CPU 1100 has a function similar to the CPU 400. The memory 1101 has a function similar to the memory 401. The communication unit 1102 has a function similar to the communication unit 402. The drive control unit 1103 has a function similar to the drive control unit 403. The automatic exposure control unit 1104 has a function similar to the automatic exposure control unit 404. The communication control unit 1105 has a function similar to the communication control unit 405.

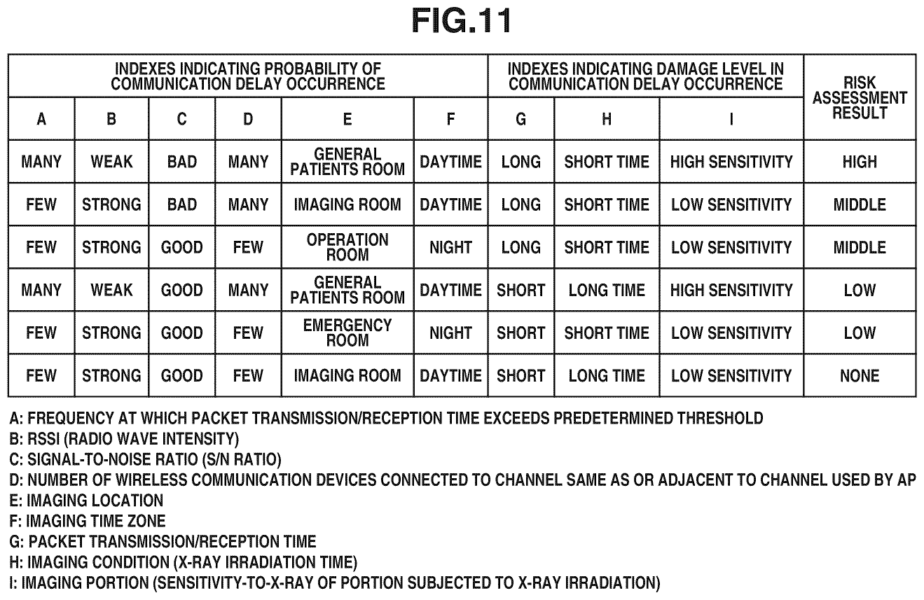

The risk assessment unit 1106 is controlled by the communication control unit 1105 and assesses a level of the risk to a subject if accuracy of stopping radiation is decreased due to occurrence of a communication delay. The risk determination unit 1107 determines the risk based on an event probability of a communication delay and a damage level in occurrence of a communication delay. While examples of index data indicating the event probability of a communication delay include a frequency of occurrence of a packet transmission and reception time period exceeding a predetermined threshold value, RSSI, the S/N ratio, the number of wireless communication devices using the same channel, an imaging location, and an imaging time zone, the present disclosure is not limited thereto. Any other indexes related to the event probability of a communication delay are also applicable. Examples of index data indicating the damage level in occurrence of a communication delay include the time period taken for packet transmission and reception, an imaging condition, and an imaging portion, the present disclosure is not limited thereto. Any other indexes indicating the damage level in occurrence of a communication delay are also applicable. FIG. 11 illustrates an example of a combination of indexes for risk assessment. For example, in a case where the event probability of a communication delay is high regardless of the imaging condition and imaging portion (for example, when a time period taken for packet transmission and reception is long and the packet transmission and reception is performed frequently), the risk assessment unit 1106 determines that the risk is high.

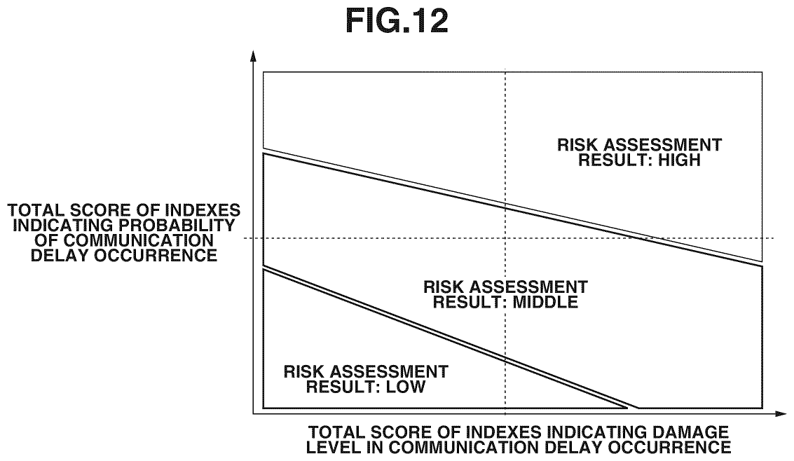

On the contrary, in a case where the event probability of a communication delay is low, the risk assessment unit 1106 determines that the risk is middle or low even with a comparatively high damage level in occurrence of a communication delay. The example in FIG. 11 is to be considered as illustrative, and index items, combinations, and risks to be assessed are not limited thereto. As illustrated in FIG. 12, scores obtained by weighting indexes each indicating the event probability of a communication delay and indexes each indicating the damage level in occurrence of the communication delay may be set, and risks may be determined based on positions of plots of the of the set total scores.

The risk determination unit 1107 is controlled by the communication control unit 1105 and determines a content of the notification based on a result of the assessment performed by the risk assessment unit 1106. More specifically, the risk determination unit 1107 manages at least one threshold value that is to be compared with a risk in occurrence of a communication delay assessed by the risk assessment unit 1106 to identify the content of the notification for the risk. Based on the plurality of threshold values managed by the risk determination unit 1107, the risk determination unit 1107 classifies a risk assessed by the risk assessment unit 1106 as one of a plurality of notification contents each indicating the suitability/unsuitability for the automatic exposure control. The threshold values that are used by the risk determination unit 1107 may be appropriately changed in response to an instruction from an apparatus in the radiation imaging system, such as the information processing apparatus 200.

The risk notification unit 1108 is controlled by the communication control unit 1105 and notifies the user of the content indicating the suitability/unsuitability for image capturing using the automatic exposure control function which has been classified by the risk determination unit 1107, as a determination result. More specifically, the risk notification unit 1108 notifies the information processing apparatus 200 of the determination result via the communication unit 1102. The information processing apparatus 200 notifies the user of a message indicating that the communication state is unsuitable for image capturing using the automatic exposure control function, as warning information for the stop control, via the display apparatus 203. In this processing for the notification, the risk notification unit 1108 changes the content of the notification to the user in accordance with one of the plurality of suitability/unsuitability states for image capturing using the automatic exposure control function determined by the risk determination unit 1107.

### Flowchart Indicating Procedure to Notify Communication State Unsuitable for Image Capturing Using Automatic Exposure Control Function

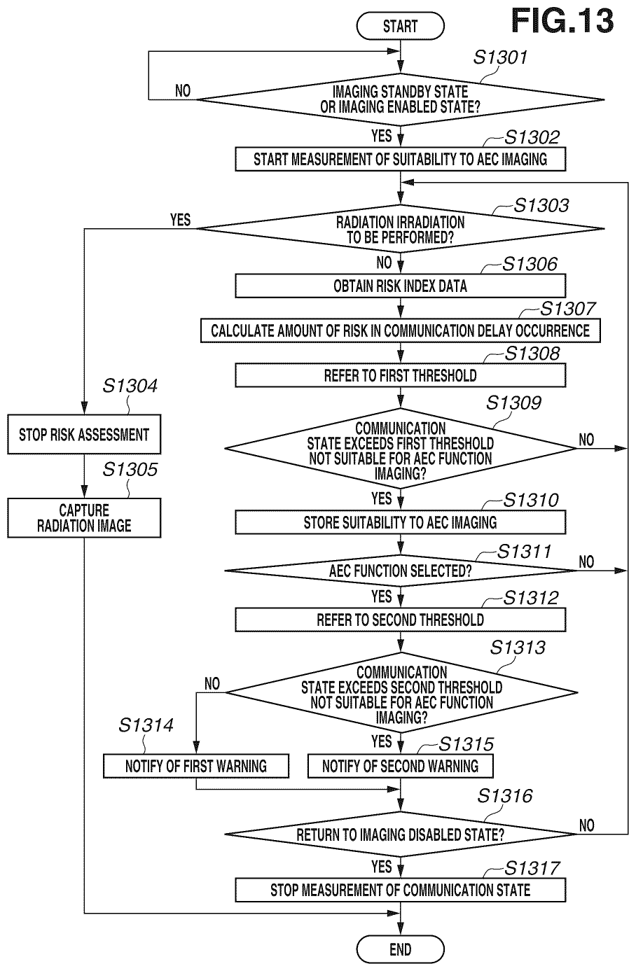

FIG. 13 is a flowchart illustrating procedure in which the radiation generation apparatus 121 changes the content of the notification to the user according to the communication state, in the radiation imaging system according to the fourth exemplary embodiment. While, in this flowchart, the risk in occurrence of a communication delay in image capturing using the automatic exposure control function is classified into three different states including the normal state, the warning state, and the error state in order of normality based on two different threshold values, the present disclosure is not limited thereto. Three or more threshold values may be used. As described above, the present exemplary embodiment uses a plurality of threshold values.

In step S1301, the radiation generation apparatus 121 performs a determination similar to the determination in step S501. In step S1301, the radiation generation apparatus 121 determines whether the radiation imaging apparatus 100 is in the imaging standby state during the transition from the imaging disabled state to the imaging enabled state or in the imaging enabled state, in which the radiation imaging apparatus 100 is ready to perform image capturing in response to receipt of radiation irradiation. In a case where the radiation generation apparatus 121 determines that the radiation imaging apparatus 100 is in either of the states (YES in step S1301), the processing proceeds to step S1302. In a case where the radiation generation apparatus 121 determines that the radiation imaging apparatus 100 is in neither of the states (NO in step S1301), the processing returns to step S1301.

Processing in step S1302 is similar to the processing in step S502. The communication control unit 1105 of the radiation generation apparatus 121 starts the risk assessment by using the risk assessment unit 1106.

In step S1303, the radiation generation apparatus 121 performs a determination similar to the determination in step S503. In step S1303, the radiation generation apparatus 121 determines whether a radiation irradiation request has been received. In a case where the radiation generation apparatus 121 determines that a radiation irradiation request has not been received and the radiation irradiation is not to be performed (NO in step S1303), the processing proceeds to step S1306. In a case where the radiation generation apparatus 121 determines that a radiation irradiation request has been received and the radiation irradiation is to be performed (YES in step S1303), the processing proceeds to step S1304.

In step S1304, to prevent occurrence of a communication noise in a radiation image, the communication control unit 1105 of the radiation generation apparatus 121 stops the risk assessment using the risk assessment unit 1106.

Processing in steps S1305 to S1307 are similar to the processing in steps S505 to S507, respectively.

In step S1308, the risk determination unit 1107 of the radiation generation apparatus 121 refers to a first threshold value. The first threshold value is used to classify the communication state as the state where the amount of risk can be ignored or the other states (warning state or error state).

In step S1309, the risk determination unit 1107 of the radiation generation apparatus 121 determines whether the communication state is unsuitable for image capturing using the automatic exposure control function (warning state or error state). In a case where the risk determination unit 1107 determines that the communication state exceeds the first threshold value, which means that the risk is in the warning state or the error state (YES in step S1309), the processing proceeds to step S1310. In a case where the risk determination unit 1107 determines that the communication state does not exceed the first threshold value, which means that risk can be ignored (NO in step S1309), the processing returns to step S1303.

Processing in steps S1310 and S1311 are similar to the processing in steps S509 and S510, respectively.

In step S1312, the risk determination unit 1107 of the radiation generation apparatus 121 refers to a second threshold value. The second threshold value is used to classify the communication state as the warning state or the error state.

In step S1313, the risk determination unit 1107 of the radiation generation apparatus 121 determines whether the risk is the warning state or the error state. In a case where the risk determination unit 1107 determines that the communication state does not exceed the second threshold value, which means that the risk is in the warning state (NO in step S1313), the processing proceeds to step S1314. In a case where the risk determination unit 1107 determines that the communication state exceeds the second threshold value, which means that the risk is in the error state (YES in step S1313), the processing proceeds to step S1315.

In step S1314, the risk notification unit 1108 of the radiation generation apparatus 121 notifies the information processing apparatus 200 that the communication state is the warning state in which the communication state exceeds the first threshold value. The information processing apparatus 200 notifies the user that the risk is at a state unsuitable for image capturing using the automatic exposure control function (first warning state), via the display apparatus 203.

In step S1315, the risk notification unit 1108 of the radiation generation apparatus 121 notifies the information processing apparatus 200 that the communication state is in the error state in which the communication state exceeds the second threshold value. The information processing apparatus 200 notifies the user that the risk is at a state unsuitable for image capturing using the automatic exposure control function (second warning state, i.e., the error state in which it is desirable that image capturing using the automatic exposure control function should be avoided), via the display apparatus 203. According to the present exemplary embodiment, the warning information to be notified is changed in accordance with the threshold value used to determine the risk.

Processing in steps S1316 and S1317 are similar to the processing in steps S512 and S513, respectively.

As described above, the radiation generation apparatus 121 of the radiation imaging system assesses a level of the risk in occurrence of a communication delay in the radiation imaging system by using the risk assessment unit 1106. Based on the plurality of managed threshold values, the risk determination unit 1107 classifies the risk as one of the plurality of states indicating the suitability/unsuitability for the automatic exposure control. The risk notification unit 1108 changes the content of the notification of the suitability/unsuitability state to the user according to the one of the plurality of suitability/unsuitability states classified by the risk determination unit 1107. This configuration allows the user to be notified of a risk increased situation, based on the content of the notification of the radiation imaging system, whereby the user can determine either to cancel image capturing using the automatic exposure control function or resolve the risk. The radiation imaging system may not permit image capturing using the automatic exposure control function in the worst risk state (third warning state). This prevents the dose of a patient from being increased due to a communication delay in the automatic exposure control before image capturing, and prevents generation of an image with a cumulative dose larger than the total cumulative dose set as a threshold value in image capturing using the automatic exposure control function.

The description has been given of the present exemplary embodiment of the present disclosure.

While the fourth exemplary embodiment is based on an example configuration in which the radiation imaging apparatus 100 includes the risk assessment unit 1106, the risk determination unit 1107, and the risk notification unit 1108, the present disclosure is not limited thereto. For example, the radiation generation apparatus 121 or the communication control apparatus 110 may include the risk assessment unit 1106, the risk determination unit 1107, and the risk notification unit 1108. While the fourth exemplary embodiment is based on a configuration in which the result of the risk determination (result of the comparison with a threshold value) is notified, the present disclosure is not limited thereto. For example, the user may be notified of only the result of the risk assessment via the communication unit, and a determination of suitability/unsuitability for image capturing using the automatic exposure control function may be left to the user.

In this case, the determination unit (the risk determination unit 1107) can be omitted, which is easier to implement the apparatus configuration (system configuration).

### Other Exemplary Embodiments

The disclosure of the present specification includes the radiation imaging system, the radiation imaging apparatus, the radiation generation apparatus, the communication control apparatus, and the information processing apparatus as follows.

### Item 1

A radiation imaging system including a radiation imaging apparatus including a detection unit configured to detect a radiation emitted from a radiation generation apparatus, and a communication unit configured to transmit information about the radiation detected by the detection unit, a control unit configured to perform stop control to stop the radiation emitted from the radiation generation apparatus, based on the information transmitted from the communication unit, a determination unit configured to determine a communication state of the communication unit, and a notification unit configured to notify of information corresponding to a result of the determination performed by the determination unit, wherein in a case where the result of the determination performed by the determination unit indicates a defective communication, the notification unit notifies of warning information for the stop control by the control unit.

### Item 2

The radiation imaging system according to item 1, wherein the determination unit determines the communication state, based on a retransmission frequency in information transmission from the communication unit.

### Item 3

The radiation imaging system according to item 1, wherein the determination unit stores a threshold value for a communication time period that is taken for the communication by the communication unit, and uses the threshold value to determine the communication state.

### Item 4

The radiation imaging system according to item 3, wherein the threshold value is changeable.

### Item 5

The radiation imaging system according to item 3 or 4, wherein the determination unit stores a plurality of the threshold values.

### Item 6

The radiation imaging system according to any one of items 3 to 5, wherein the communication unit performs a first communication at a plurality of times before radiation irradiation, and the determination unit determines the communication state based on a comparison between a statistical value of a communication time period of the first communication performed at the plurality of times and the threshold values.

### Item 7

The radiation imaging system according to any one of items 3 to 5, wherein the communication unit performs the first communication at a plurality of times before radiation irradiation, and the determination unit determines a defective communication based on the number of times when a communication time period of the first communication exceeds the threshold value.

### Item 8

The radiation imaging system according to any one of items 1 to 5, wherein in a case where a result of the determination of the communication state by the determination unit changes from a favorable communication to a defective communication, the notification unit notifies of the warning information.

### Item 9

The radiation imaging system according to any one of items 3 to 5, wherein the notification unit changes the warning information to be notified of in accordance with the threshold value, among the plurality of threshold values, that has been used by the determination unit in the determination of the communication state.

### Item 10

A radiation imaging apparatus including a detection unit configured to detect a radiation emitted from a radiation generation apparatus, a control unit configured to control whether the detection unit performs an operation detecting the radiation during the radiation irradiation, a communication unit configured to transmit information about the radiation detected by the detection unit during the radiation irradiation, and a determination unit configured to determine a communication state of the communication unit, wherein in a case where a result of the determination performed by the determination unit indicates a defective communication, the communication unit transmits warning information for the operation detecting the radiation during the radiation irradiation performed by the detection unit.

### Item 11

A radiation generation apparatus for emitting a radiation toward a radiation imaging apparatus that performs an operation detecting the radiation emitted during radiation irradiation and transmits information about the detected radiation, the radiation generation apparatus includes a determination unit configured to determine a communication state of the radiation imaging apparatus, and a communication unit configured to transmit information corresponding to the result of the determination performed by the determination unit, wherein in a case where the result of the determination performed by the determination unit indicates a defective communication, the communication unit transmits warning information for the operation detecting the radiation emitted during the radiation irradiation performed by the radiation imaging apparatus.

### Item 12

A communication control apparatus for communicating with a radiation imaging apparatus that performs an operation detecting the radiation emitted during radiation irradiation and transmits information about the detected radiation, the communication control apparatus includes a determination unit configured to determine a communication state of the radiation imaging apparatus, and a communication unit configured to transmit information corresponding to a result of the determination performed by the determination unit, wherein in a case where the result of the determination performed by the determination unit indicates a defective communication, the communication unit transmits warning information for the operation detecting the radiation emitted during the radiation irradiation performed by the radiation imaging apparatus.

### Item 13

An information processing apparatus for receiving and processing radiation image information from a radiation imaging apparatus that performs an operation detecting a radiation emitted during radiation irradiation and transmits information about the detected radiation, the information processing apparatus includes a determination unit configured to determine a communication state of the radiation imaging apparatus, and a communication unit configured to transmit information corresponding to a result of the determination performed by the determination unit, wherein in a case where a result of the determination performed by the determination unit is a defective communication, the communication unit transmits warning information for the operation detecting the radiation emitted during the radiation irradiation performed by the radiation imaging apparatus.

### Item 14

A radiation imaging system including a radiation imaging apparatus including a detection unit configured to detect a radiation emitted from a radiation generation apparatus, and a communication unit configured to transmit information about the radiation detected by the detection unit, a control unit configured to perform stop control to stop the radiation emitted from the radiation generation apparatus, based on the information transmitted from the communication unit, a detection unit configured to detect a communication state of the communication unit, and a notification unit configured to notify of information corresponding to a result of the detection performed by the detection unit.

### Item 15

A radiation imaging apparatus including a detection unit configured to detect a radiation emitted from a radiation generation apparatus, a control unit configured to control whether the detection unit performs an operation detecting the radiation during the radiation irradiation, a communication unit configured to transmit information about the radiation detected by the detection unit during the radiation irradiation, and a detection unit configured to detect a communication state of the communication unit, wherein the communication unit transmits information corresponding to a result of the detection performed by the detection unit.

### Item 16

A radiation imaging system including a radiation imaging apparatus including a detection unit configured to detect a radiation emitted from a radiation generation apparatus, and a communication unit configured to transmit information about the radiation detected by the detection unit, a control unit configured to perform stop control to stop the radiation emitted from the radiation generation apparatus, based on the information transmitted from the communication unit, a determination unit configured to obtain a risk index from the communication unit and determine a risk of a decrease in accuracy of the stop control that is performed by the control unit, and a notification unit configured to notify of information corresponding to a result of the determination performed by the determination unit, wherein in a case where the result of the determination performed by the determination unit indicates a defect, the notification unit notifies of warning information for the stop control that is performed by the control unit.

### Item 17

The radiation imaging system according to item 16, wherein the determination unit determines, based on a risk index based on either or both of an index indicating an event probability of a communication delay in a communication from the communication unit and an index indicating an influence in occurrence of the communication delay, a risk level due to the communication delay.

### Item 18

The radiation imaging system according to item 17, wherein the index indicating the event probability of the communication delay includes threshold values that are related to a frequency of occurrence of a time period for communication exceeding a predetermined threshold value, RSSI, S/N ratio, and a number of wireless communication devices connected to a channel same as or adjacent to a channel used by an access point, and determines the risk level due to the communication delay by using the threshold values.

### Item 19

The radiation imaging system according to item 17, wherein the index indicating the influence in occurrence of the communication delay includes threshold values that are related to a time period of communication, an imaging condition, an imaging location, and an imaging time zone, and determines the risk level due to the communication delay by using the threshold values.

### Item 20

The radiation imaging system according to any one of items 18 and 19, wherein the threshold values are changeable.

### Item 21

The radiation imaging system according to any one of items 18 and 19, wherein the determination unit includes a plurality of the threshold values.

### Item 22

The radiation imaging system according to any one of items 18 to 21, wherein the communication unit performs the first communication at a plurality of times before radiation irradiation, and wherein the determination unit determines the risk level based on the index indicating the event probability of the communication delay obtained in the first communication and the index indicating the influence in occurrence of the communication delay.

### Item 23

The radiation imaging system according to any one of items 16 to 21, wherein in a case where a result of the determination of the risk level performed by the determination unit changes from favorable to defective, the notification unit notifies of the warning information.

### Item 24

The radiation imaging system according to any one of items 18 to 21, wherein the notification unit changes the warning information to be notified in accordance with the threshold value, among the plurality of the threshold values, used by the determination unit in the determination of the risk level.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD) TM), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Applications No. 2023-084772, filed May 23, 2023, and No. 2024-017342, filed Feb. 7, 2024, which are hereby incorporated by reference herein in their entirety.

What is claimed is:

1. A radiation detection system that performs stop control to stop radiation irradiation of a radiation generation apparatus, based on a result of dose detection performed by a radiation detection apparatus, the radiation detection system involving a communication between the radiation detection apparatus and the radiation generation apparatus via a network in an execution of the stop control, the radiation detection system comprising:
a notification device configured to notify a user of information; and
one or more controllers configured to perform functions including
determining a communication state of the network; and
issuing a predetermined notification based on a result of the determination,
wherein the notification device notifies of notification information for the stop control, based on the predetermined notification.

2. The radiation detection system according to claim 1, wherein the determination is based on a retransmission frequency in the communication via the network.

3. The radiation detection system according to claim 1, wherein the determination is based on a comparison between a threshold value for a communication time period and an actual communication time period between the apparatuses.

4. The radiation detection system according to claim 3, wherein the threshold value is changeable.

5. The radiation detection system according to claim 3, wherein a plurality of values is stored as the threshold value.

6. The radiation detection system according to claim 3, wherein the actual communication time period is a statistical value obtained in a plurality of communications.

7. The radiation detection system according to claim 1, wherein, in the determination, a comparison between a plurality of communication time periods obtained in the plurality of communications and the threshold value is performed at a plurality of times, and based on a number of times of communication that satisfies a predetermined condition with respect to the threshold value, a determination of whether the network is defective is performed.

8. The radiation detection system according to claim 1, wherein, in a case where the communication state changes from favorable to defective, warning information is notified as the notification information.

9. The radiation detection system according to claim 5, wherein a content of the notification information is changed in accordance with a value, among the plurality of values stored as the threshold value, used for the determination.

10. The radiation detection system according to claim 5, wherein, based on at least either one of an index indicating an event probability of a communication delay and an index indicating an influence in occurrence of the communication delay, the one or more controllers control the notification device to notify of information about a risk level due to the communication delay as the notification information.

11. The radiation detection system according to claim 10, wherein the index indicating the event probability of the communication delay includes threshold values that are related to a frequency of occurrence of a communication time period exceeding a threshold value, received signal strength indication (RSSI), signal-to-noise (S/N) ratio, and a number of wireless communication devices connected to a channel same as or adjacent to a channel used by an access point.

12. The radiation detection system according to claim 10, wherein the index indicating the influence in occurrence of the communication delay includes threshold values that are related to a communication time period, an imaging condition, an imaging location, and an imaging time zone.

13. A control device included in a radiation detection system that performs stop control to stop radiation irradiation of a radiation generation apparatus based on a result of dose detection performed by a radiation detection apparatus, the radiation detection system involving a communication between the radiation detection apparatus and the radiation generation apparatus via a network in execution of the stop control, the control device comprising:
one or more controllers configured to perform functions including
determining a communication state of the network; and
issuing a predetermined notification based on a result of the determination,
wherein a notification device notifies of notification information for the stop control based on the predetermined notification.

14. An information processing apparatus including a control device included in a radiation detection system that performs stop control to stop radiation irradiation of a radiation generation apparatus based on a result of dose detection performed by a radiation detection apparatus, the radiation detection system involving a communication between the radiation detection apparatus and the radiation generation apparatus via a network in execution of the stop control, the control device comprising:
one or more controllers configured to perform functions including
determining a communication state of the network; and
issuing a predetermined notification based on a result of the determination,
wherein a notification device notifies of notification information for the stop control based on the predetermined notification.

15. The information processing apparatus according to claim 14, wherein the information processing apparatus is either a radiation detection apparatus or a radiation generation apparatus.
